(19) United States
(12) Patent Application Publication
(10) Pub. No.: US 2005/0245737 A1
(43) Pub. Date: Nov. 3, 2005

(54) Apparatus and method for mixed-bed lectin chromatography

(76) Inventor: Richard D. Cummings, Edmond, OK (US)

Correspondence Address: DUNLAP, CODDING & ROGERS P.C., PO BOX 16370, OKLAHOMA CITY, OK, 73113, US

(21) Appl. No.: 11/111,322

(22) Filed: Apr. 21, 2005

Related U.S. Application Data
(60) Provisional application No. 60/564,435, filed on Apr. 22, 2004.

(51) Int. Cl. C07H 5/04; C07H 5/06; C08B 37/00

(52) U.S. Cl. 536/55.3; 536/124; 210/656

(57) ABSTRACT

The present invention contemplates an apparatus and method for isolating glycoconjugates from mixtures or contaminated mixtures thereof. In the present invention, a bed or other support element comprising a mixture of immobilized lectins is provided. The mixture of glycoconjugates is passed over the mixed lectin bed wherein glycoconjugates which correspond to the lectins in the bed are bound thereto while non-glycoconjugates flow away. Thus, the method can be used to isolate glycoproteins from mixtures of glycoproteins and non-glycosylated proteins, glycopeptides from mixtures of glycopeptides and non-glycosylated peptides, glycolipids from non-glycosylated lipids, and free oligosaccharides from extracts or preparations. This invention solves the problem of isolating glycoconjugates from complex mixtures of glycoconjugates with non-gyconconjugates. For example, in most cells, a large fraction of the total macromolecules are not glycosylated. Glycomics and glycoproteomics specifically are concerned with macromolecules which contain carbohydrates. Thus, the mixed bed lectin chromatography described herein will expand both glycomics and glycoproteomics, which are currently hampered by lack of methods or devices or approaches able to be used to generally isolate all or most of the glycoconjugates in cells or extracts of cells in a simple and direct approach that has few steps.

### CROSS-REFERENCE TO RELATED APPLICATIONS

This application claims the benefit under 35 U.S.C. 119(e) of U.S. Provisional Application Ser. No. 60/564,435, filed Apr. 22, 2004, the contents of which are hereby expressly incorporated by reference herein in its entirety.

### BACKGROUND

There are currently several crude and relatively inefficient methods for use in isolating glycoconjugates. One current state-of-the art method for isolating glycoconjugates includes the use of absorption or chromatography of mixtures containing glycoconjugates on individual lectins. Lectins are carbohydrate-binding proteins found in plants, animals, and microorganisms. Lectins are classified into a small number of specificity groups, including for example, mannose, galactose, N-acetylglucosamine, N-acetylgalactosamine, L-fucose and N-acetylneuraminic acid, according to the monosaccharide which is the most effective inhibitor of the agglutination of erythrocytes or precipitation of polysaccharides or glycoproteins by the particular lectin. The lectins within each group may differ markedly in their affinity for the specific monosaccharide or its derivatives. Moreover, certain lectins combine more strongly with di-, tri, and tetra-saccharides than with monosaccharides. In such oligosaccharides, the specific monosaccharide is usually present at the nonreducing end, although there are lectins that react with internally placed sugars as well. The use of lectins to bind glycoconjugates has been exploited for many years, as evidenced in a very early publication of Goldstein, who used the plant lectin concanavalin A to isolate polysaccharides (1). Since the early 1980's this type of approach has been expanded by Cummings and is called serial lectin affinity chromatography, wherein a series of lectins are used in separate chromatographic steps to isolate specific glycoconjugates. Other major references describe the use of immobilized lectins to isolate glycoconjugates and even intact cells (6-17).

However, all of these references cite the use of a single type of free or immobilized lectin for isolating or characterizing a glycoconjugate. This type of an approach does not allow the widespread isolation of general glycoconjugates, but is restricted to those that are specifically recognized by the particular lectin used. The method is thus limited in its potential use and value.

### SUMMARY OF THE INVENTION

The present invention contemplates an apparatus and method for isolating glycoconjugates from mixtures or contaminated mixtures thereof. In the present invention, a bed or other support element comprising a mixture of immobilized lectins is provided. The mixture of glycoconjugates is passed over the mixed lectin bed wherein glycoconjugates which correspond to the lectins in the bed are bound thereto while non-glycoconjugates flow away. Thus, the method can be used to isolate glycoproteins from mixtures of glycoproteins and non-glycosylated proteins, glycopeptides from mixtures of glycopeptides and non-glycosylated peptides, glycolipids from non-glycosylated lipids, and free oligosaccharides from extracts or preparations. This invention solves the problem of isolating glycoconjugates from complex mixtures of glycoconjugates with non-gyconconjugates. For example, in most cells, a large fraction of the total macromolecules are not glycosylated. Glycomics and glycoproteomics specifically are concerned with macromolecules which contain carbohydrates. Thus, the mixed bed lectin chromatography described herein will expand both glycomics and glycoproteomics, which are currently hampered by lack of methods or devices or approaches able to be used to generally isolate all or most of the glycoconjugates in cells or extracts of cells in a simple and direct approach that has few steps.

### DESCRIPTION OF THE DRAWINGS

FIG. 1 is a schematic diagram of an embodiment of the invention.

### DESCRIPTION OF THE INVENTION

The present invention contemplates an apparatus and method for isolating glycoconjugates from mixtures or contaminated mixtures thereof. In the present invention, a mixture of lectins is provided on a bed or other support element. The lectins may be derivatized with fluorescent dyes, gold particle, biotin, or enzymes in manners known by those of ordinary skill in the art. Preferably, the mixed-bed lectin chromatography (MBLC) described herein comprises a mixture of at least two or more immobilized lectins (including, but not limited to those listed herein) for isolating glycoconjugates (e.g., glycoproteins, glycopeptides, glycolipids, glycosaminoglycans, and free oligosaccharides) which comprise one or more of the carbohydrate or monosaccharide components fucose (Fuc), galactose (Gal), N-acetylglucosamine (GlcNAc), N-acetylgalactosamine (GalNAc), mannose (Man), glucose 9 (Glc), and sialic acids, and derivatives thereof. MBLC allows the separation of glycosylated molecules from non-glycosylated molecules in mixtures of the two types. Thus, the method can be used to isolate glycoproteins from mixtures of glycoproteins and proteins, glycopeptides from mixtures of glycopeptides and peptides, and free oligosaccharides from extracts or preparations. MBLC will be highly advantageous to modern biochemical approaches, including those recognized as proteomic, glycoproteomic, and glycomic. For example, MBLC can enable the isolation and/or separation of the “glycome” from cell and tissue extracts. The glycome is a term analogous to the terms that characterize the genome and proteome, wherein “glycome” is defined as the total carbohydrate complement and cells, tissues, and/or organisms.

Various mixed-bed lectins could be prepared containing, for example, mixtures of two different immobilized lectins (e.g., Con A and GSL-I-B₄), three different immobilized lectins (e.g., Con A, GSL-I-B₄, and RCA-I), four different immobilized lectins, e.g., WFA, UEA-I, WGA, and GSL-II (or others listed above or elsewhere herein), or potentially up to dozens of different immobilized lectins.

In one embodiment, the present invention comprises an apparatus or method having or using any combination of at least two, three, four, five, six, seven, eight, nine, ten, eleven, twelve, thirteen, fourteen, fifteen, sixteen, seventeen, eighteen, or more lectins including but not limited to any two or more of the lectins listed anywhere herein. Other lectins not listed herein can also be used as long as the apparatus or method functions in accordance with the present invention.

For example, lectins which may be used in the embodiments of the present invention are listed in the Handbook of Plant Lectins: Properties and Biomedical Applications, by Els J. M. Van Damme, Willy J. Peumans, Arpad Pusztai, Susan Bardocz. New York, N.Y., John Wiley & Sons, 1998. 452p., the entirety of which is expressly hereby incorporated by reference herein.

Other lectins which may be used in the present invention, as noted above, are shown in Appendix I herein.

The mixed-bed lectins may be contained in microcolumns (e.g., with dimensions 1 mm×10 mm) or in larger columns (e.g., with dimensions 1 cm mm×100 cm) for either gravity or high-pressure-type chromatography or in a fluidized bed or other applicable chromatographic apparatus or methods known to those of ordinary skill in the art.

The lectins can be covalently immobilized on solid-type supports, which include but are not limited to, Ultralink™, Aminolink™, Affi-Gel™, w-Aminohexyl™, Carbolink™, Diaminopropyl, Adipic Acid Hydrazide, Sulfalink™, Thio Propyl Sepharose™, Thiol Sepharose™, Thiol Propyl, Affarose™, and CNBr-Sepharose™. Or, the lectins can be non-covalently immobilized on a support element, such as by using biotinylated lectins captured non-covalently on immobilized Streptavidin or Avidin. In general, the lectins will be immobilized on a chromatographic resin. The lectins described herein are known by persons of ordinary skill in the art and are commercially available, however, it is contemplated that the apparatus and method of the present invention may also comprise or use molecules categorized as lectins but which are not yet described or available but which may be made available in the future.

Mixtures (e.g., cell extracts, aspirates; sera, biological fluids) of glycoconjugates and potentially non-glycoconjugates, are passed over the column (or other support element) wherein materials not bound by the lectins can be removed by washing with simple buffers using compositions and methods known by those of ordinary skill in the art and as discussed elsewhere herein.

Glycoconjugates which are bound by the lectins can be eluted, for example, with a buffer comprising a mixture of lectin-binding haptens, i.e., a “mixed hapten buffer”. The mixed hapten buffer preferably comprises a mixture of monosaccharides (or oligo/polysaccharides) that could include, for example, alone or in combination, the following: fucose, mannose, α-methyl-mannose, GlcNAc, GalNAc, galactose, lactose, raffinose, stachyose, glucose, sialic acids, chitobiose, chitotriose, chitotetraose, and maltose.

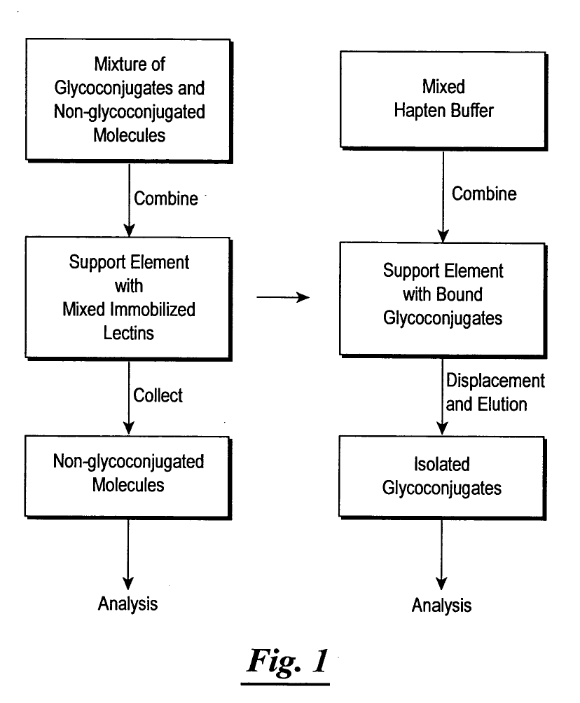

In a typical embodiment of the present invention as represented in FIG. 1, at least two different types of lectins are immobilized on a support material such as described elsewhere herein. A mixture of molecules, obtained from any source, containing glycoconjugates and non-glycosylated molecules is passed over the mixed lectin bed comprising the support material and the lectins. The glycoconjugates bind to the lectins of the mixed lectin bed and the non-glycosylated molecules pass over and through the mixed lectin bed and are collected in a collection vessel. This initial eluate containing the non-glycosylated molecules can then be further analyzed if desired, for example by mass spectrometry or other methods known in the art. Then, the mixed lectin bed is saturated with a mixed hapten buffer comprising various saccharides as described elsewhere herein which bind to the lectins on the mixed lectin bed thereby displacing the glycoconjugates on the mixed lectin bed. Employing gravity flow, the displaced glyconjugates are eluted into another collection vessel. The elected glycoconjugates can then be further analyzed using methods known in the art. The support material may be disposed within a column for example.

In an alternative embodiment of the invention, rather than using a column, a combination of the mixed-bed lectin supports or matrices can be provided in a suspension and used in solution to adsorb the target glycoconjugates from the mixture being purified. The adsorbed glycoconjugates on the lectin support could then be separated from the solution of unadsorbed material by gravity sedimentation or filtration, for example, and the adsorbed glycoconjugates on the immobilized lectins could be eluted or separated from the matrices using the mixed hapten buffer described previously.

In one embodiment, the mixed lectin bed comprises from two to nine of the lectins listed in Table I. In another embodiment, the mixed lectin bed comprises from two to 14 of the lectins listed in Table II. In another embodiment, the mixed lectin bed comprises from two to all 23 of the lectins listed in both Table I and Table II. In another embodiment, the mixed lectin bed comprises two or more of the lectins listed in Appendix I herein.

TABLE I
Multiple Lectin Set.

| | Corresponding |
|---|---|
| | Lectin-binding |
| Lectin | Carbohydrate |
| Canavalia ensiformis (Con A) | Man-rich N-glycans |
| | & terminal Glc-R |
| Griffonia simplicifolia lectin-I-B₄ (GSL-I-B₄) | terminal Galα1-3-R |
| Griffonia simplicifolia lectin-II (GSL-II) | terminal |
| | GlcNAcα/β1-R |
| Ricinus communis-I (RCA-I) | terminal |
| | Galβ4GlcNAc-R |
| Triticum vulgaris (Wheat Germ Agglutinin-WGA) | terminal Sialic |
| | acid-R and |
| | GlcNAc-R |
| Ulex europaeus (UEA-I) | terminal |
| | Fucα1-2Galβl1-R |
| Wisteria floribunda agglutinin (WFA) | terminal |
| | GalNAcα/β-R |
| Aleuria aurantia Lectin (AAL) | terminal Fuc-R |
| Limax flavus agglutinin (LFA) | terminal |
| | Sialic acid-R) |

TABLE II
Multiple Lectin Set

| | Corresponding |
|---|---|
| | Lectin-binding |
| Lectin | carbohydrate |
| Anguilla anguilla (eel lectin) | (Fucα1-2 and Fucα1-4) |
| Arachis hypogaea (peanut agglutinin) | (Galβ3GalNAcα1-Ser/Thr) |
| Datura stramonium (jimson weed) | (Galβ4GlcNAc)n-R) |
| Erythrinia cristagalli (coral tree lectin) | (Galβ4-R) |
| Helix pomatia (edible snail) | (GalNAcα1-R) |
| Lotus tetragonolobus (lotus lectin) | (Fucα1-3/4GlcNAc-R) |
| Lycopersicon esculentum (tomato lectin) | (Galβ4GlcNAc)n-R) |
| Maackia amurensis (MAL or MAA) | (Sialic |
| | acidα2-3Galβ4GlcNAc-R) |
| Phaseolus vulgaris (L-PHA) | (tri/tetraantennary N-glycans) |
| Phaseolus vulgaris (E-PHA) | (bisected biantennary |
| | N-glycans) |
| Pisum sativum (pea lectin) | (core fucosylated tri/bi |
| | N-glycans) |
| Sambucus nigra (bark lectin) | (Sialic |
| | acidα2-6Gal/Gal/GalNAc) |
| Solanum tuberosum (potato lectin) | (long chain |
| | (Galβ4GlcNAc)n-R) |
| Dolichos biflorus (horse gram) | (GalNAcα1-R) |

### Utility

The mixed-bed lectin chromatography method described herein, wherein two or more lectins are used together in a chromatographic step followed by exposure to a mixed hapten buffer to elute bound glycoconjugates from the MBLC is previously unknown. This new method avoids multiple extra steps necessary for serial chromatography on lectins in separate chromatographic supports thereby allowing a robust technique to isolate many different glycoconjugates in a single step. The development of MBLC will now allow the field of glycomics to develop by making possible the direct isolation of multiple glyconjugates in a single step from complex mixtures of material derived from cells, tissues, organs, fluids, organisms, or extracts thereof.

This invention solves the problem of isolating glycoconjugates from complex mixtures of glycoconjugates with non-gyconconjugates. For example, in most cells, a large fraction of the total macromolecules are not glycosylated. Glycomics and glycoproteomics specifically are concerned with macromolecules which contain carbohydrates. Thus, MBLC will expand the fields of both glycomics and glycoproteomics, which are currently hampered by lack of methods or devices or approaches able to be used to generally isolate all or most of the glycoconjugates in cells or extracts of cells in a simple and direct approach that has few steps.

The use of MBLC will have an impact in many areas of medical research and basic science, where glycoconjugates are thought to play important roles, but in which the basic structures of the glycoconjugates and the macromolecules containing attached carbohydrates are poorly defined. These poorly defined roles include, but are not limited to, the following: cancer, including cancer initiation, cancer progression, cancer diagnosis, and cancer prognosis; immunology, including the innate immune system and the adaptive immune system, where carbohydrate-containing macromolecules, including receptors and antibodies, are thought to play key roles in immune regulation; parasitology, wherein parasites present a large array of glycoconjugates that are both immunogenic in the infected animal, but which are also useful to parasites in their adaption and survival in the infected hosts; inflammatory diseases and lymphocyte homing, wherein glycoconjugates on circulating cells and the lining of blood vessels play key roles in cellular adhesion and cell signaling; development and birth defects, wherein there are many changes in glycoconjugate structure and metabolism, as yet poorly defined, and defects in these changes due to genetic mutations cause abnormal development.

Among the advantages of the present invention are: (1) it is the first comprehensive method and apparatus for the isolation of glycoconjugates in complex mixtures by a single step procedure; (2) it's easy to use, thereby allowing commonly skilled workers to perform isolations of glycoconjugates; (3) low costs of materials and reagents; (4) simplicity in use; (5) speed of isolation of glycoconjugates; and (6) utility in a variety of conditions, including harsh detergents, some quantities of organic solvents, high salt, protein extracts and denaturants (including chaotropes, such as guanidinium hydrochloride).

The present invention is not to be limited in scope by the specific embodiments described herein, since such embodiments are intended as but single illustrations of one aspect of the invention and any functionally equivalent embodiments are within the scope of this invention. Indeed, various modifications of the methods of the invention in addition to those shown and described herein will become apparent to those skilled in the art form the foregoing description.

Each of the references, patents or publications cited herein is incorporated by reference in its entirety.

### Cited References

1. Goldstein, I. J., and So, L. L. (1965) Arch Biochem Biophys 111, 407-414
2. Cummings, R. D., and Kornfeld, S. (1982) J Biol Chem 257, 11235-11240
3. Cummings, R. D., Kornfeld, S., Schneider, W. J., Hobgood, K. K., Tolleshaug, H., Brown, M. S., and Goldstein, J. L. (1983) J Biol Chem 258, 15261-15273
4. Cummings, R. D., and Kornfeld, S. (1984) J Biol Chem 259, 6253-6260
5. Merkle, R. K., and Cummings, R. D. (1987) Methods Enzymol 138, 232-259
6. Lis, H., and Sharon, N. (1998) Chem Rev 98, 637-674
7. Lis, H., and Sharon, N. (1986) Annu Rev Biochem 55, 35-67
8. Sharon, N., and Lis, H. (1972) Science 177, 949-959
9. Sharon, N., and Lis, H. (1986) Nature 323, 203-204
10. Sharon, N., and Lis, H. (1989) Science 246, 227-234
11. Sharon, N., and Lis, H. (1990) Faseb J 4, 3198-3208
12. Sharon, N., and Lis, H. (1993) Sci Am 268, 82-89
13. Sharon, N., and Lis, H. (2001) Adv Exp Med Biol 491, 1-16
14. Sharon, N., and Lis, H. (2002) J Agric Food Chem 50, 6586-6591
15. Nicolson, G. L., and Poste, G. (1979) Biochim Biophys Acta 554, 520-531
16. Lotan, R., and Nicolson, G. L. (1979) Biochim Biophys Acta 559, 329-376
17. Reading, C. L., Belloni, P. N., and Nicolson, G. L. (1980) J Natl Cancer Inst 64, 1241-1249

### Appendix I

A
AAA—Allium ascalonicum agglutinin (shallot)
AAA—Aloe arborescens agglutinin (Kidachi aloe, narrow leaved sword aloe)
AAA—Artocarpus altilis agglutinin
AAA, AAnA—Anguilla anguilla agglutinin (freshwater eel, European eel)
AAurA—Aleuria aurantia agglutinin (orange peel fungus)
AAusA—Androctonus australis agglutinin (Saharan scorpion)
ABA, AbiA, ABL—Agaricus bisporus agglutinin (mushroom)
ABrA—Amphicarpaea bracteata agglutinin (hog peanut)
ACA—Allium cepa agglutinin (onion)
ACA—Alocasia indica lectin
ACA—Amaranthus caudatus agglutinin (amaranth, tassel flower, inca wheat)
ACL—Amaranthus cruentus lectin (red amaranth, purple amaranth)
ACmA—Arisaema curvatum lectin
AFA—Afimbrial adhesin (bacteria)
AGG—Agrocybe ylindracea (mushroom, fruiting bodies)
AGL—Aplysia gonad lectin
AIA—Artocarpus integrifolia agglutinin (Artocarpus heterophyllus, jaca, Indian jaca tree, jackfruit)
AIRM1—adhesion inhibitory receptor molecule, Siglec-7, I-type lectin from NK cells, monocytes
ALA—Artocarpus lakoocha agglutinin (lakoocha, small jack, monkey fruit)
AlloA—Allomyrina dichtoma agglutinin
AAnA, AAA —Anguilla anguilla agglutinin (eel)
AMA—Allium moly agglutinin (dwarf flowering onions)
AMA—Arum maculatum agglutinin (lords and ladies)
AQN—spermadhesin
APA—Aaptos papillata agglutinin
APA—Abrus precatorius agglutinin (jequirity bean, coral bead plant, lucky bean, crab's eyes)
APA/APL—Aegopodium podagraria agglutinin/lectin (ground elder, achweed)
APA—Allium porrum agglutinin (leek)
APL—Aquathanatephorus pendulus lectin
ARA—Agropyrum repens agglutinin (couch grass)
AREL—Agropyrum repens embryo lectin (couch grass)
ARL—Athelia rolfsii lectin
ARLL—Agropyrum repens leaf lectin (couch grass)
ASA/ASL—Allium sativum agglutinin/lectin (garlic, garden rocambole)
ASGP-R—asialoglycoprotein receptor
ASL—Amaranthus spinosus agglutinin (thorny pigweed, spiny amaranth)
AUA—Allium ursinum agglutinin (ramson, bears garlic)
AVA—Allium vineale agglutinin (crow garlic)
AWN—spermadhesin

B
BanLec—Banana lectin (Musa paradisiac)
BCL—Botrytis cinerea lectin
BDA—Bryonia dioica agglutinin (white bryony)
BfL—Butea frondosa lectin (Butea monosperma, bastard teak, flame of the forrest)
BGA—Biomphalaria glabrata agglutinin
Blec—bud lectin (Pisum sativum)
BLA—Birgus latro agglutinin (coconut crab)
BMA—Bowringia milbraedii agglutinin
BPA—Bauhinia purpurea agglutinin (camels foot tree, purple mountain ebony)
BSA/BSL/BSI/BSII—Bandeiraea simplicifolia agglutinin/lectin/isolectin (Griffonia simplicifolia)
BsyL—Brachypodium sylvaticum lectin (false brome grass)

C
C-14—Gallus gallus, chicken lectin
C-16—Gallus gallus, chicken lectin
CA—Cymbidium agglutinin
CAA—Caragana arborescens agglutinin (Siberian pea tree)
CAA/CPA—Cicer arietinum agglutinin (chick pea, ceri bean)
CAA/CAL—Colchicum autumnale agglutinin/lectin (meadow saffron)
Calsepa—Calystegia sepium agglutinin
CBL—Cyphomandra betacea lectin (tamarillo gruit, tree tomato)
CBP-35—Lactosamine-binding protein (mouse fibroblasts)
CBP-67—Carbohydrate-binding protein (rat liver nuclei)
CBP-70—Carbohydrate-binding protein (HL60 cell nuclei)
CCL—Ceratobasidium cornigerum lectin
CD22—Siglec-2, I-type lectin from B cells
CD33—Siglec-3, I-type lectin from myeloid progenitors, mature monocytes
CD-MPR—Cation dependent mannose 6-phosphate receptor
CRD—carbohydrate recognition domain
CEA—Colocasia esculenta lectin (taro)
CEL—Cucumaria echinata (sea cucumber) lectin
CGA—Canavalia gladiata lectin (Japanese Jack bean)
CGA—Canna generalis lectin
CHA—Cepaeae hortensis agglutinin (snail)
CHA—Cymbidium hybrid lectin
CIA—Coccinia grandis lectin (C. indica, C. cordifolia, Ivy gourd, scarlet gourd)
CI-MPR—Cation independent mannose-phosphate receptor
CLA—Cladrastis lutea lectin (Yellow wood)
CLA—Clivia miniata agglutinin (Clivia)
CLC—Charcot-Leyden crystal protein
CLL—Chicken lactose-binding lectin
CMA—Chelidonium majus agglutinin (celandine, greater celandine)
CMA—Clivia miniata lectin
CMA—Cucurbita maxima agglutinin (great pumpkin, common gourd, marrow, winter squash)
CMA—Cytisus multiflorus agglutinin
CNX—calnexin
Con A—Concanavalin A (Canavalia ensiformis, jack bean)
Conarva—Convolvulus arvensis agglutinin
ConBr—Concanavalin Br (Canvalia brasiliensis)
ConGF—Canavalia grandiflora agglutinin
CPC/CAA—Cicer arietinum agglutinin (chick pea, ceri bean)
CPA—Cucurbita pepo agglutinin (pumpkin, summer squash, gourd)
CRA—Carcinoscorpin (Carcinoscorpius rotunda)
CRCA—Carcinoscorpius rotunda cauda (Indian horseshoe crab)
CRT—calreticulin
CS, CSA, CSA-II, CSL—Cytisus scoparius agglutinin (Sarothamnus scoparius, busch broome, Scotch broom)
CSA, CSA-I, CSL—Cytisus sessilifolius agglutinin (Portugal broome)
CSL—Cerebellar soluble lectin, cell-sealing lectin
CTA—Clerodendron trichotomum lectin
CTL—Croton tiglium lectin (croton)

D
DBA—Dolichos biflorus agglutinin (horse gram)
DC-SIGN (or CD209)—external C-type lectin at the surface of both mature and immature dendritic cells
DEC-205—C-type lectin from dendritic cells
DGA—Dioclea grandiflora lectin
DguiL—Dioclea guianensis lectin
DIA—Datura innoxia agglutinin
DLA, LPA—Dolichos lablab agglutinin (Lablab niger, Lablab purpureus, Hyacinth bean, lablab bean, black seeded kidney bean)
Dmgal—Drosophila tandem repeat galectin
DSA—Datura stramonium agglutinin (Jimson weed, thornapple)

E
EBL—Elderberry lectin (Sambucus nigra agglutinin (elderberry, eldertree, elder) Sambucus nigra, eldertree, elder)
ECA, ECorA—Erythrina corallodendron agglutinin (West Indian coral tree)
ECA, ECL—Erythrina cristagalli agglutinin (cocks comb coral tree)
EEA—Euonymus europaeus agglutinin (prickwood, spindle tree)
EHA—Epipactis helleborine agglutinin (broad leaved helleborine)
EHA, EHL—Eranthis hyemalis lectin (winter aconite)
EHA—Euphorbia heterophylla agglutinin (Mexican fire plant, painted spurge)
Endo 180/uPARAP—urokinase-type plasminogen activator receptor associated protein
ERGIC-53—mammalian intracellular lectin with similarity to legume lectins

F
FSL—Fucose-specific lectin

G
Gal—galectin, beta-galactoside specific lectins in vertebrates, invertebrates, sponge and fungus
GBL—Glucan-binding lectin (Streptococcus sp.)
GCA—Geodia cydonium agglutinin
GC1, GC2—Geodia cydonium galactins
GMP-140—Platelet granule membrane protein-140, p-selectin
GNA—Galanthus nivalis agglutinin (snowdrop)
GNL—Peanut nodule lectin (Arachis hypogaea)
GPA—Gonatanthus pumilus agglutinin
GS, GSA—Griffonia simplicifolia agglutinin (now Bandeirea simplicifolia agglutinin)
GSL—Gerardia savaglia lectin (false foxglove)

H
HAA—Helix aspersa agglutinin (garden snail)
HAA—Homarus americanas agglutinin (lobster)
HARE—hyaluronic acid receptor for endocytosis
HBL—Human brain lectin—
HCA—Hura crepitans agglutinin (sand-box tree)
Heltuba—Helianthus tuberosus agglutinin (Jerusalem artichoke), see also HTA
HHA—Hippeastrum hybrid agglutinin (amaryllis)
HL-3, HL-13—Human lectins
HL-29—Lactosamine-binding protein (human lung)
HPA—Helix pomatia agglutinin (Roman snail, edible snail)
HTA—Helianthus tuberosus lectin (Jerusalem artichoke) see also Heltuba
HVA—Hordeum vulgare lectin (barley)

I
IAA—Iberis amara agglutinin (candy tuft)
IGF-II/MPR—insulin-like growth factor II mannose-6-phosphate receptor
IRA—Iris hybrid lectin (Dutch iris)
ix-bp—Coagulation factor ix-binding protein from the venom of habu snake

J
JFL—Jacalin, Jackfruit lectin (Artocarpus heterophyllus) (bread fruit tree)
JRL—Jacalin related lectin

K
KLR, KLRG1—killer cell-lectin like receptor G1

L
L-I, L-II—Leaf lectins from Winged bean (Psophocarpus tetragonolobus, goa bean, winged pea)
L-29, HL-29—lactosamine-binding protein (human lung)
L-34—beta-galactoside-specific lectin (mouse fibrosarcoma)
LAA, LAL, LALA—Laburnum alpinum agglutinin (Scotch laburnum)
LAA—Leptospermum archinoides agglutinin (Australian tea tree)
LAA—Leucojum aestivum agglutinin (snowflake, summer snowflake)
LAA—Luffa acutangula agglutinin (ridge gourd)
LAF Limulus 18-kDa agglutination-aggregation factor
LAL, LAA, LALA—Laburnum alpinum agglutinin (Scotch laburnum)
LAL—Laelia auatumnalis lectin
LAM-14—Mouse lymphocyte homing receptor
LANA—Laburnum angyriodes agglutinin (laburnum)
LBA, LBL, PLA—Lima bean agglutinin (Phaseolus limensis, Phaseolus lunatus)
LBP—Laminin-binding protein (mouse macrophages)
LCA, LcH—Lens culinaris agglutinin (lentil)
LcH, LCA—Lens culinaris agglutinin (lentil)
LCL—Litchi chinensis lectin
LcLI, II—Lathyrus cicera isolectins (dwarf chicling vetch, vetch)
LEA, LEL, TL—Lycopersicon esculentum agglutinin (tomato)
LEC-1, Lec-1 etc—nematode galectins
LEC-CAM—Selectins, group of C-type lectins
LEL—Loranthus europaeus lectin (loranthus, misteltoe)
LEL, LEA, TL—Lycopersicon esculentum agglutinin (tomato)
LFA—Limax flavus agglutinin
LL1 Lymphocyte lectin 1 (mammals)
LNA—Lablab niger agglutinin
LOA—Lathyrus odoratus lectin (sweet pea)
LOA1, 2—Listera ovata (twayblade)
LoLI, II—Lathyrus ochrus isolectins (yellow flowered pea)
LPA, DLA—Lablab purpureus agglutinin (Lablab niger, Dolichos lablab, Hyacinth bean, lablab bean, black seeded kidney bean)
LPA—Lathyrus pratensis agglutinin (bastard vetchling, meadow lathyrus)
LPA—Limulin (Limulus polyphemus, horseshoe crab)
LSA—Lathyrus sativum agglutinin (chicling vetch)
LTA—Lotus tetragonolobus agglutinin (lotus, birds foot treefoil, also Tetragonolobus purpurea, winged pea, asparagus pea)
LtubL—Lathyrus tuberosus tuber lectin (tuberous lathyrus)
LtuLI, II—Lathyrus tuberosus seed isolectins (tuberous lathyrus)
LVA—Leucojum vernum agglutinin (snowflake, spring snowflake)

M
M6P receptors—mannose-6-phosphate receptors, P-type lectin
Mac-2—Macrophoage surface antigen, major non-integrin laminin-binding protein (man, mouse)
MAA, MAH, MAHs, MAL—Maackia amurensis agglutinin/lectin
MAG—Siglec4, I-type lectin from oligodendrocytes, Schwann cells
MAH, MAHs, MM, MAL—Maackia amurensis agglutinin/lectin
MAL, MM, MAH, MAHs—Maackia amurensis agglutinin/lectin
MBA—Machaerium biovulatum agglutinin
MBA—Mung bean agglutinin (Vigna radiata, Phaseolus aureus)
MBL—mannose-binding lectin, also MBP
MBP—Maltose/mannose/maltose-binding protein (animals)
MBP-A—Mannose-binding protein A (rat)
MCA—Momordica charantia agglutinin (bitter pear melon, bitter gourd)
ME-C2, ME-D2, ME-E2, ME-F2—Machaerocereus eruca isolectins
MEA—Machaerocereus eruca lectin
MEL-14—Mouse lymphocyte homing receptor
MGA—Mycoplasma gallisepticum agglutinin
mGBP—Mouse galactose binding protein
MIA—Mangifera indica agglutinin (mango tree)
MIS—myeloid inhibitory Siglec
ML, VAA—Mistletoe lectin (Viscum album)
MLA—Macharium lunatus agglutinin
MLL—Mulberry leaf lectin
MMA, MML—Marah macrocarpus lectin (wild cucumber)
MMR—Macrophage mannose receptor (animals)
MNL—Peanut nodule and cotyledon lectin (Arachis hypogea)
MPA—Maclura pomifera agglutinin (maclura, osage orange, hedge apple tree)
MPR—mannose 6-phosphate receptor
mSiglec—mouse Siglec
MT LEC1—Medicago truncatala lectin

N
NFA—Nonfimbrial adhesin (bacteria)
NFL—Neoregelia flandria lectin
NLA—Narcissus lobularis agglutinin
NPA/NPL—Narcissus pseudonarcissus agglutinin/lectin (daffodil)

O
OB-BP1—obesity binding protein, Siglec-6, I-type lectin from B-cells, placental trophoblasts
OCIL—Osteoclast inhibitory lectin
OSA, RL—Oryza sativa agglutinin (rice)

P
PA-I, PA-II—Pseudomonas aeruginosa lectins
Pa-1,2,3,4,5—Phytolacca americana isolectins (pokeweed, pigeon berry)
PAA, Pa-1,2,3,4,5—Phytolacca americana isolectins (pokeweed, pigeon berry)
PAA—Percea americana agglutinin (avocado)
PALL—Phragmites australis lectin (common reed)
PADGEM—Platelet granule membrane protein-140, p-selectin
PCA—Phaseolus coccineus agglutinin (scarlet runner bean)
PFA—snail lectin
PHA—Phytohemagglutinin (Phaseolus vulgaris, red kidney bean)
PHA-E—Erythroagglutinating isolectin of PHA
PHA-L—Leucoagglutinating isolectin of PHA
PL—Pseudomonas lectin
PL-A, PL-B, PL-C, PL-D, PA, PAA—
PLA, LBA, LBL—Phaseolus limensis agglutinin (P. lunatus, lima bean)
PMA—Polygonatum multiflorum lectin (common Solomon's seal)
PNA—Arachis hypogaea agglutinin (peanut)
po66-CBP—Beta-galactoside-binding lectin in lung carcinoma
PPA—Ptilota plumosa agglutinin (red marine algae)
PRA—Peanut root lectin (Arachis hypogea)
PRA—Pterocarpus rhorii agglutinin
PSA, PsA—Pisum sativum agglutinin (garden pea, common pea)
PsNlec-1—Pisum sativum nodule lectin 1 (garden pea, common pea)
PTA, PTL, WBA—Psophocarpus tetragonolobus agglutinin (goa bean, winged pea)
PVL—Psathylera velutina lectin
PWM—Poke weed mitogen (Phytolacca americana)

R
R1—Receptro 1, recognin 1
RAGE—receptor for advanced glycation endproducts
RaRF—Ra reactive factors (mammalian serum)
RCA, RCA120, RCL I, RCL II—Ricinus communis agglutinin (castor oil bean)
RCA60, RCL III, RCL IV—ricin, ricin D, ricin E (Ricinus communis, castor bean, ricin)
RCL—Rhizoctonia crocorum lectin
RHL—rat hepatic lectin
RL, OSL—Rice lectin (Oryza sativa)
RL-29—Lactosamine-binding protein (rat lung)
RPA, RPsA—Robinia pseudoaccacia seed agglutinin (black locust, false acacia)
RpbA—Robinia pseudoaccacia bark agglutinin (black locust, false acacia)
RSA—Rhizoctonia solani lectin

S
SAF-2—Siglec-8, I-type lectin from eosinophils, mast cells
SAL—Sialic acid specific lectin—
SAP—Serum amyloid protein (mammals)
SBA—Soybean agglutinin (Glycine max, soya bean)
SCA—Sambucus canadensis lectin (Canadian elderberry)
SCA—Secale cereale lectin (rye)
SEA—Sambucus ebulus lectin (dward elder)
SER—Sheep erythrocyte receptor (mouse macrophages)
SGA—Sauromatum guttatum agglutinin
SGL—Sarcocystis gigantea lectin
SHA—Salvia horminum lectin (salvia)
Siglec-1—sialoadhesin from macrophages, I-type lectin
Siglec-2—CD22, I-type lectin from B cells
Siglec-3—CD33, I-type lectin from myeloid progenitors, mature monocytes
Siglec-4—MAG, I-type lectin from oligodendrocytes, Schwann cells
Siglec-5—I-type lectin from monocytes, neutrophils
Siglec-6—OB-BP1, I-type lectin from B-cells, placental trophoblasts
Siglec-7—AIRM1, I-type lectin from NK cells, monocytes
Siglec-8—SAF-2, I-type lectin from eosinophils, mast cells
Siglec-9—I-type lectin from monocytes, neutrophils, NK cells (subset)
Siglec-10—I-type lectin from B cells, eosinophils, monocytes
Siglec-11—I-type lectin
SJA or SJAbg/SJAbm—Sophora japonica agglutinin (Japanese/Chinese pagoda tree)—
SL—Onobrychis viciifolia lectin (sanfoin)
SML—Sarcocystis muris lectin
SML—Sclerotinia minor lectin
SNA—Sambucus nigra agglutinin (elderberry, eldertree, elder)—
SP-A—Pulmonary surfactant protein-A (mammals)
SP-D—surfactant protein-D
SRA—Sambucus racemosa lectin (red-berried elder)
STA—Solanum tuberosum agglutinin (potato)
SSA—Salvia sclarea agglutinin (clary, fetid clary sage)
SSA—Sambucus sieboldiana lectin (Japanese elderberry)
SSA—Soybean seedling agglutinin
SSA—Stenostylis stenocarpa agglutinin
SML—Scierotinia sclerotiorum lectin
SVAK—Snake venom agglutinin (Naja naja kaouthia)
SVAM—Snake venom agglutinin (Naja mossambica mossambica)
SWA—Sarothamnus welwitschii lectin (broom)

T
TAA—Thorn apple agglutinin (Datura stramonium, Jimson weed)
TCA—Tetracarpidium conophorum lectin (Nigerian walnut)
TKA—Trichosantes kirilowii agglutinin (serpent cucumber)
TL, LEA, LEL—Tomato lectin (Lycopersicon esculentum)
TL, TxLC, TXLM—Tulipa lectins (tulip)
TPA—Tetragonolobus purpurea agglutinin (winged pea, asparagus pea, also Lotus Tetragonolobus, lotus, birds foot treefoil)
TxLC-I, TL—Tulipa lectin (tulip)
TXLM-I, TXLM-II—Tulipa lectins (tulip)

U
UDA—Urtica dioica agglutinin (stinging nettle, nettle)
UEA—Ulex europaeus agglutinin (furze, gorse)

V
VAA, ML—Viscum album agglutinin (mistletoe)
VCA—Vicia cracca lectin (common vetch)
VEA—Vicia ervilia lectin (bitter vetch)
VFA—Favin, Vicia faba agglutinin (broad bean, garden bean)
VGA—Vicia graminea agglutinin
VIP-36—mammalian intracellular lectin with similarity to legume lectins
VRA—Vigna racemosa agglutinin
VSA—Vicia sativa agglutinin (tare, vetch)
VVA, VVL—Vicia villosa agglutinin (hairy vetch)

W
WBA, PTA, PTL—Winged bean agglutinin (Psophocarpus tetragonolobus, goa bean, winged pea)
WBTL—Winged bean tuber lectin (Psophocarpus tetragonolobus, goa bean, winged pea)
WGS-I—Winged bean green shell lectin (Psophocarpus tetragonolobus, goa bean, winged pea)
WFA, WFH—Wisteria floribunda agglutinin (Japanese wisteria)
WGA—Wheat germ agglutinin (Triticum vulgare)

X
XL35—Xenopus laevis oocyte lectin

Z
ZMA—Zea mays lectin (corn, maize)

1. A method of separating glycosylated molecules from non-glycosylated molecules, comprising:
providing a support element having at least two types of lectins immobilized on a support material;
providing a mixture of molecules comprising glycosylated molecules and non-glycosylated molecules;
combining the mixture of molecules with the support element having the at least two types of immobilized lectins;
removing the glycosylated molecules which are bound to the at least two types of immobilized lectins on the support element by washing the support element with a mixed hapten buffer comprising one or more saccharides which bind to the at least two types of immobilized lectins on the support element thereby displacing the glycosylated molecules from the at least two types of immobilized lectins; and
collecting the glycosylated molecules which are eluted from the support element due to displacement from the at least two types of immobilized lectins by the mixed hapten buffer.

2. The method of claim 1 comprising the additional step of collecting the non-glycosylated molecules which pass over the support element without binding to the at least two types of immobilized lectins after combining the mixture of molecules with the support element.

3. The method of claim 1 wherein in the step of providing the support element, the at least two types of immobilized lectins comprise at least one of fucose, galactose, N-acetylglucosamine, N-acetylgalactosamine, mannose, glucose, sialic acids, and derivatives thereof.

4. The method of claim 1 wherein in the step of providing a support element, the at least two types of immobilized lectins are selected from the groups of lectins in Table I and Table II.

5. The method of claim 1 wherein in the step of providing a support element, the at least two types of immobilized lectins are selected form the lectins in Appendix I.

6. The method of claim 1 wherein in the step of providing the support element, the support element comprises a chromatography column.

7. The method of claim 1 wherein in the step of removing the glycosylated molecules, the mixed hapten buffer comprises at least one saccharide selected from the group consisting of fucose, mannose, α-methyl-mannose, GlcNAc, GalNAc, galactose, lactose, raffinose, stachyose, glucose, sialic acids, chitobiose, chitotriose, chitotetraose, and maltose.

8. The method of claim 1 wherein in the step of providing a mixture of molecules comprising glycosylated molecules, the glycosylated molecules comprise glycoproteins, glycopeptides, glycolipids, glycosaminoglycans, free oligosaccharides, and/or polysaccharides, or other glycoconjugates.

9. A chromatography column comprising a support element comprising two or more different lectins immobilized thereon.

10. The chromatography column of claim 9 wherein the two or more lectins immobilized thereon are selected from the lectins listed in Appendix I, Table I, and Table II.

11. A method of separating glycosylated molecules from non-glycosylated molecules, comprising:
providing a support material having at least two types of lectins immobilized thereon;
providing a mixture of molecules comprising glycosylated molecules and non-glycosylated molecules;
combining the mixture of molecules with the support material having the at least two types of immobilized lectins; and
removing the glycosylated molecules which are bound to the at least two types of immobilized lectins on the support material by washing the support material with a mixed hapten buffer comprising one or more saccharides which bind to the at least two types of immobilized lectins on the support material thereby displacing the glycosylated molecules from the at least two types of immobilized lectins on the support material.

12. The method of claim 11 comprising the additional step of collecting the glycosylated molecules which are eluted from the support material due to displacement from the at least two types of immobilized lectins by the mixed hapten buffer.

13. The method of claim 11 comprising the additional step of collecting the non-glycosylated molecules which pass over the support material without binding to the at least two types of immobilized lectins after combining the mixture of molecules with the support material.

14. The method of claim 11 wherein in the step of providing the support material, the at least two types of immobilized lectins comprise at least one of fucose, galactose, N-acetylglucosamine, N-acetylgalactosamine, mannose, glucose, sialic acids, and derivatives thereof.

15. The method of claim 11 wherein in the step of providing a support material, the at least two types of immobilized lectins are selected from the groups of lectins in Table I and Table II.

16. The method of clam 11 wherein in the step of providing a support material, the at least two types of immobilized lectins are selected form the lectins in Appendix I.

17. The method of claim 11 wherein in the step of providing the support material, the support material comprises a portion of a chromatography column.

18. The method of claim 11 wherein in the step of removing the glycosylated molecules, the mixed hapten buffer comprises at least one saccharide selected from the group consisting of fucose, mannose, α-methyl-mannose, GlcNAc, GalNAc, galactose, lactose, raffinose, stachyose, glucose, sialic acids, chitobiose, chitotriose, chitotetraose, and maltose.

19. The method of claim 11 wherein in the step of providing a mixture of molecules comprising glycosylated molecules, the glycosylated molecules comprise glycoproteins, glycopeptides, glycolipids, glycosaminoglycans, free oligosaccharides, and/or polysaccharides, or other glycoconjugates.

20. A chromatography apparatus comprising a support material comprising two or more different lectins immobilized thereon.

21. The chromatography apparatus of claim 20 wherein the two or more lectins immobilized thereon are selected from the lectins listed in Appendix I, Table I, and Table II.
